(19) United States
(12) Patent Application Publication
(10) Pub. No.: US 2005/0124843 A1
(43) Pub. Date: Jun. 9, 2005

(54) Method and apparatus for delivering targeted therapy to a patient

(75) Inventor: Anurag K. Singh, St. Louis, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(73) Assignee: Washington University

(21) Appl. No.: 10/731,663

(22) Filed: Dec. 9, 2003

(51) Int. Cl. A61N 5/00; A61M 29/00

(52) U.S. Cl. 600/3; 604/96.01

(57) ABSTRACT

An applicator for delivering targeted radiation brachytherapy to tissue adjacent a cavity of a patient. The applicator includes a balloon adapted for introduction to the cavity of the patient, wherein the balloon has a deflated state in which the balloon is adapted for insertion into the cavity and an inflated state in which the balloon is enlarged for at least partially filling the cavity of the patient. The balloon moves from the deflated state to the inflated state upon introduction of pressurized fluid to an interior of the balloon. The applicator also includes a conduit in fluid communication with the interior of the balloon for introducing pressurized fluid to the interior of the balloon to move the balloon from the deflated state to the inflated state, and a catheter extending over at least a portion of the balloon for delivering radiation from a radiation source to the tissue adjacent the cavity.

### BACKGROUND OF THE INVENTION

The present invention relates generally to delivering targeted therapy to a patient, and more specifically to delivering targeted therapy to tissue adjacent a cavity of the patient.

In radiation brachytherapy, catheters are placed in close proximity to the tissue targeted for radiation. Currently, such proximity is achieved by free-hand placement of brachytherapy catheters over a needle. However, such free-hand placement is challenging and can vary greatly between operators, making it difficult to consistently achieve accurate placement of the catheter. Moreover, a variety of organs are inaccessible for free-hand placement of brachytherapy catheters and therefore are not routinely treated with brachytherapy. Single balloon catheters have been used for brachytherapy to treat breast cancer using a radiation source positioned within the center of the balloon. However, controlling the distribution of radiation to the target tissue as well as achieving a quick fall-off of dose to the non-target tissue can be difficult because of the single dwell position of the radiation source and because of the distance between the source and the target tissue.

### SUMMARY OF THE INVENTION

In one aspect, the present invention includes an applicator for delivering targeted radiation brachytherapy to tissue adjacent a cavity of a patient. The applicator includes a balloon adapted for introduction to the cavity of the patient, wherein the balloon has a deflated state in which the balloon is adapted for insertion into the cavity and an inflated state in which the balloon is enlarged for at least partially filling the cavity of the patient. The balloon moves from the deflated state to the inflated state upon introduction of pressurized fluid to an interior of the balloon. The applicator also includes a conduit in fluid communication with the interior of the balloon for introducing pressurized fluid to the interior of the balloon to move the balloon from the deflated state to the inflated state, and a catheter extending over at least a portion of the balloon for delivering radiation from a radiation source to the tissue adjacent the cavity.

In another aspect, the present invention includes an applicator system for delivering targeted thermal therapy to tissue adjacent a cavity of a patient. The applicator system includes a balloon adapted for introduction to the cavity of the patient, wherein the balloon has a deflated state in which the balloon is adapted for insertion into the cavity and an inflated state in which the balloon is enlarged for at least partially filling the cavity of the patient. The balloon moves from the deflated state to the inflated state upon introduction of pressurized fluid to an interior of the balloon. The applicator system also includes a conduit in fluid communication with the interior of the balloon for introducing pressurized fluid to the interior of the balloon to move the balloon from the deflated state to the inflated state, and a catheter extending over at least a portion of the balloon. The catheter has a heat source therein for delivering heat to the tissue adjacent the cavity.

In yet another aspect, a method is provided of delivering targeted radiation brachytherapy to tissue adjacent a cavity of a patient using an applicator including a balloon having a deflated state in which the balloon is adapted for insertion into the cavity of the patient and an inflated state in which the balloon is enlarged for at least partially filling the cavity. The method includes attaching a catheter to the balloon for movement with the balloon, inserting the balloon and the catheter into the cavity when the balloon is in the deflated state, inserting a radiation source into the catheter so the radiation source is generally adjacent the balloon, inflating the balloon within the cavity so the radiation source is a predetermined distance from the tissue adjacent the cavity, and controlling a dose distribution of radiation delivered into the tissue adjacent the cavity by the radiation source by controlling the predetermined distance of the radiation source from the tissue.

In even another aspect, a method is provided of delivering targeted thermal therapy to tissue adjacent a cavity of a patient using an applicator including a balloon having a deflated state in which the balloon is adapted for insertion into the cavity of the patient and an inflated state in which the balloon is enlarged for at least partially filling the cavity. The method includes attaching a catheter to the balloon for movement with the balloon, inserting the balloon and the catheter into the cavity when the balloon is in the deflated state, inserting a heat source into the catheter so the heat source is generally adjacent the balloon, inflating the balloon within the cavity so the heat source is a predetermined distance from the tissue adjacent the cavity, and controlling a temperature increase of the tissue adjacent the cavity by controlling the predetermined distance of the heat source from the tissue.

In yet another aspect, the present invention includes an applicator system for facilitating the delivery of at least one of external beam radiation and external thermal therapy to tissue adjacent a cavity of a patient. The applicator system includes a balloon adapted for introduction to the cavity of the patient, wherein the balloon has a deflated state in which the balloon is adapted for insertion into the cavity and an inflated state in which the balloon is enlarged for at least partially filling the cavity of the patient. The balloon moves from the deflated state to the inflated state upon introduction of pressurized fluid to an interior of the balloon. The applicator system also includes a conduit in fluid communication with the interior of the balloon for introducing pressurized fluid to the interior of the balloon to move the balloon from the deflated state to the inflated state, and a catheter extending over at least a portion of the balloon. The catheter has a radio opaque maker therein adjacent the balloon for marking a position of the balloon when the balloon is received within the cavity.

Other features of the present invention will be in part apparent and in part pointed out hereinafter.

### BRIEF DESCRIPTION OF THE DRAWINGS

FIG. 1 is a perspective of an applicator of the present invention including a balloon shown in a deflated state;

FIG. 2 is a partially cut-away perspective of the applicator of FIG. 1 showing the balloon in an inflated state;

FIG. 3 is a partially cut-away perspective of a cavity of a patient and an applicator system of the present invention for delivering targeted radiation brachytherapy to tissue adjacent the cavity;

FIG. 4 is a partially cut-away perspective of a cavity of a patient and an applicator system of the present invention for delivering targeted thermal therapy to tissue adjacent the cavity;

FIG. 5 is a partially cut-away perspective of a cavity of a patient and an applicator system of the present invention for facilitating the delivery of external beam radiation to tissue adjacent the cavity; and

FIG. 6 is a perspective of an alternative embodiment of the applicator of the present invention.

Corresponding reference characters indicate corresponding parts throughout the several views of the drawings.

### DETAILED DESCRIPTION OF THE PREFERRED EMBODIMENT

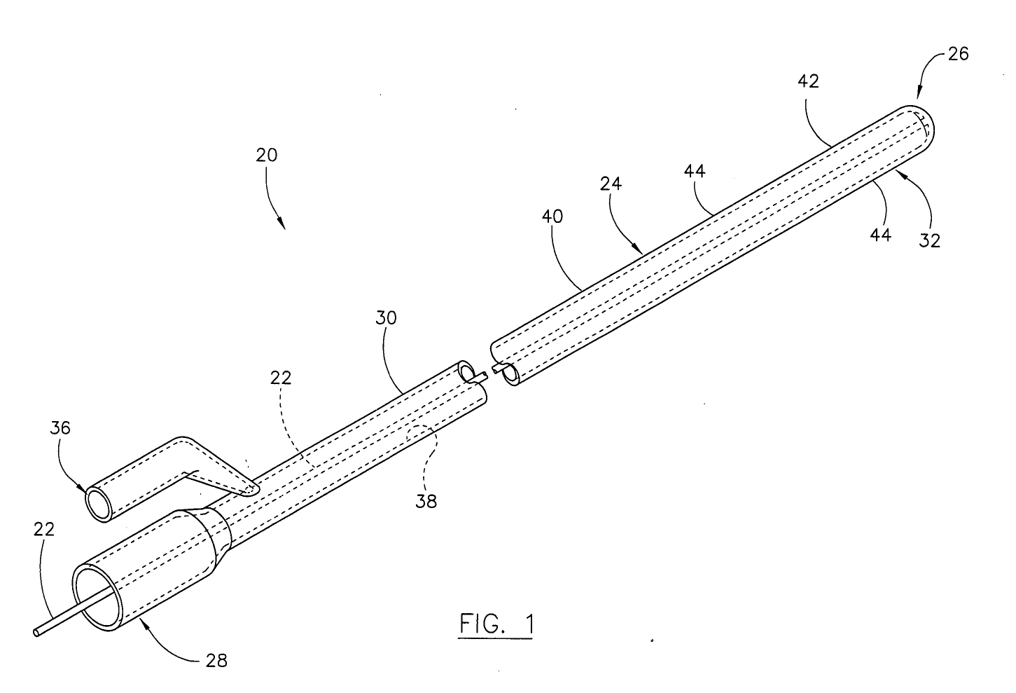

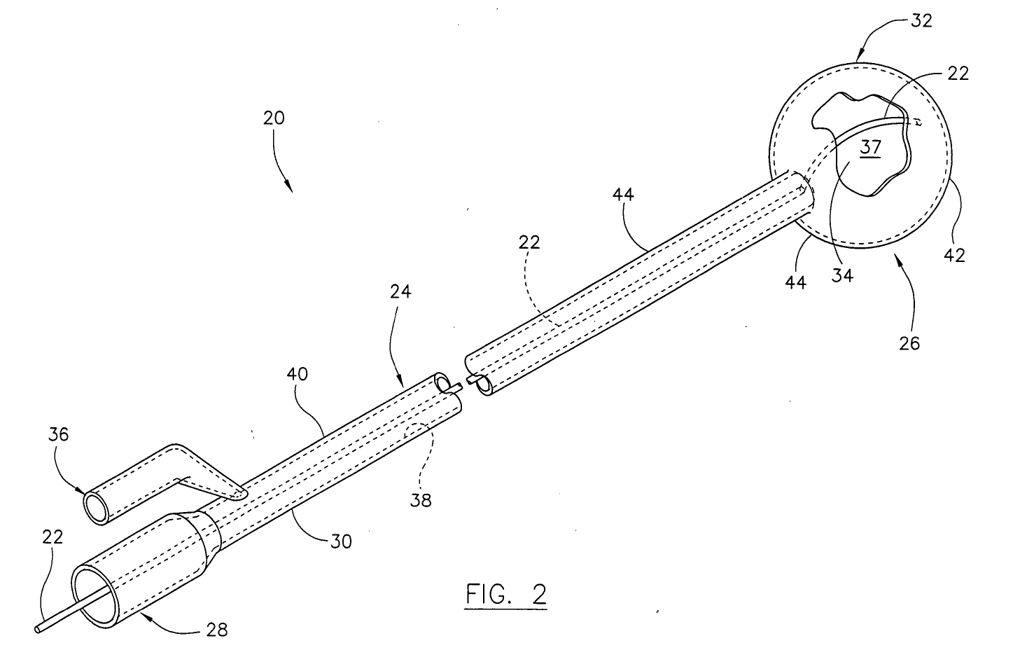

Referring now to the drawings, and more specifically to FIGS. 1 and 2, an applicator is designated in its entirety by the reference numeral 20. The applicator 20 includes a catheter 22 and a body (generally designated by 24) having a first end (generally designated by 26), a second end (generally designated by 28), a conduit 30 extending between the first end and the second end, and a balloon (generally designated by 32).

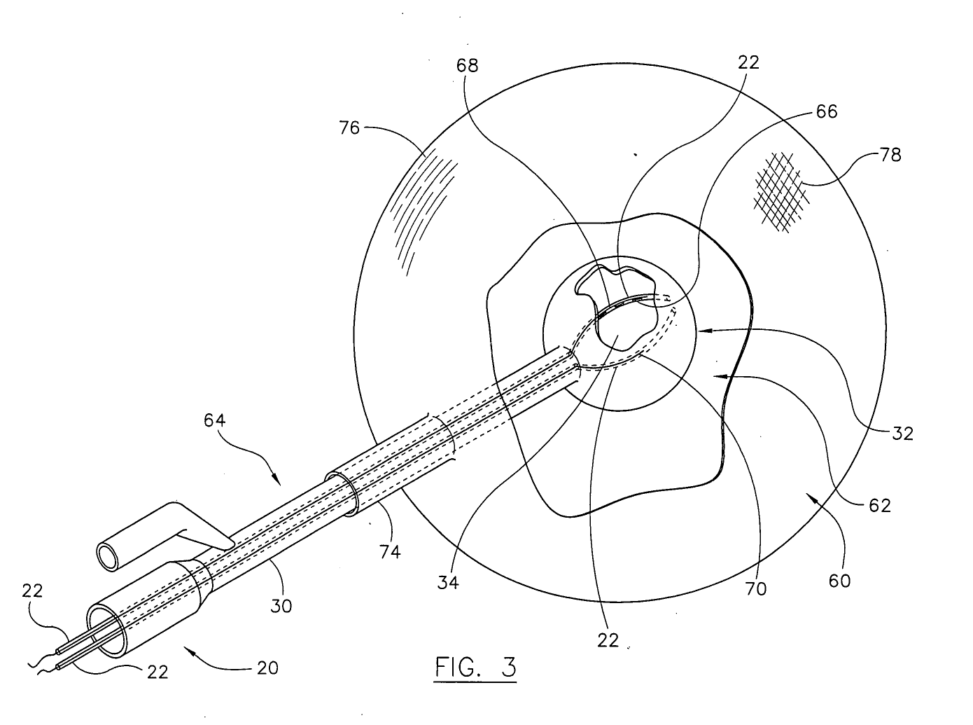

The balloon 32 is adapted for introduction to a cavity (designated by 62 in FIG. 3) of a patient, such as a patient's bladder, esophagus, and/or rectum. More specifically, the balloon 32 has a deflated state (FIG. 1) in which the balloon and the first end 26 of the body 24 are adapted for insertion into the cavity through an entrance to the cavity. Additionally, at least a portion of the conduit 30 may also be adapted for insertion through the entrance and into the cavity. The first end 26 of the body 24, the balloon 32 in its deflated state, and where applicable all or a portion of the conduit 30, are sized and shaped appropriately for insertion into the particular cavity (e.g., bladder) through its entrance (e.g., urethra). The balloon 32 also has an inflated state (FIG. 2) in which the balloon is enlarged for at least partially filling the cavity. The conduit 30 is in fluid communication with an interior 34 of the balloon 32 for introducing pressurized fluid to the interior of the balloon to move (inflate) the balloon 32 from the deflated state to the inflated state. Pressurized fluid is introduced into the conduit 30 through an opening (generally designated by 36) within the body 24 in fluid communication with the conduit. Although any suitable fluid (e.g., saline) may be introduced into the conduit 30 and the interior 34 of the balloon to move the balloon from the deflated state to the inflated state without departing from the scope of the present invention, in one embodiment air is used to move the balloon from the deflated state to the inflated state.

As illustrated in FIGS. 1 and 2, the balloon 32 defines the first end 26 of the body 24. However, the balloon 32 may be suitably positioned anywhere along the body 24 such that the balloon is adapted for insertion into the cavity and for movement (inflation) to the inflated state once received within the cavity. Additionally, in the inflated state the balloon 32 may be suitably shaped for the particular cavity. For example, the balloon 32 may be generally spherical in the inflated state when the balloon is intended to at least partially fill a patient's bladder, or may be generally cylindrical in the inflated state when the balloon is intended to at least partially fill a patient's rectum or esophagus. The particular size (e.g., radius) of the balloon 32 in the inflated state may also vary depending upon the particular cavity in which it is intended to be used. Additionally, the balloon 32 may be inflatable to a variety of sizes and/or shapes such that the inflated state of the balloon may comprise a plurality of states each having a different size and/or shape.

The body 24 may be formed from any suitable material(s), for example rubber and/or plastic. Although different sections of the body 24 may be formed from different materials, in one embodiment the entirety of the body is formed from one material. The portions of the body 24 adapted for introduction to the patient's cavity may be formed from any material suitable for use within the cavity, so that such portions do not damage tissue adjacent the cavity and/or injure/infect the patient. Additionally, in one embodiment at least a portion of the body 24 (e.g., at least a portion of the conduit 30 and/or the balloon 32) is formed from a transparent material to facilitate use of a viewing apparatus (70, FIG. 3) with the applicator 20, as is described in more detail below.

The catheter 22 extends over at least a portion of the balloon 32 and is adapted for movement with the balloon as the balloon is moved from the deflated state to the inflated state. Although the applicator 20 is described herein and illustrated in FIGS. 1, 2, and 6 as including only one catheter 22, it should be understood that the applicator 20 may include a plurality of catheters as is illustrated in FIGS. 3-5. The size of the entrance to the cavity may influence the maximum number of catheters included with the applicator 20. As Illustrated in FIGS. 1 and 2, in one embodiment the catheter 22 extends through the conduit 30 and along an interior surface 37 of the balloon 32, and is attached to the interior surface for movement with the interior surface when the balloon is moved from the deflated state to the inflated state. The catheter 22 may also be attached to an interior surface 38 of the conduit 30. In an alternative embodiment, the catheter 22 extends along an exterior surface 40 of the conduit 30 and/or an exterior surface 42 of the balloon. In yet another alternative embodiment, the catheter extends within the body 24, and more specifically within a wall 44 of the body defining at least one of the conduit 30 and the balloon 32. As will be described in more detail below, the catheter 22 is adapted to receive a device (not shown in FIGS. 1 and 2) facilitating treatment of the tissue adjacent the patient's cavity.

As illustrated in FIG. 3, the applicator 20 described above may be used to deliver targeted radiation brachytherapy to tissue (generally designated by 60) adjacent a patient's cavity (generally designated by 62). More specifically, an applicator system (generally designated by 64) includes the applicator 20 and a radiation source (generally designated by 66) in the catheter 22. Each catheter 22 may include any number of radiation sources 66. Although other radiation sources may be used without departing from the scope of the present invention (e.g., radioactive ribbons, radioactive pellets), in the exemplary embodiment illustrated in FIG. 3 the radiation source 66 is a radioactive seed 66 attached to a wire 68. The seed may be formed from any suitable radioactive isotope, such as Iridium 192, Cesium 137, Iodine 125, and/or Palladium 103. The wire 68 is positioned in the catheter 22 so the seed 66 is generally adjacent the balloon 32. The applicator system 64 may also include a viewing apparatus 70 positioned generally adjacent the balloon 32 for viewing the catheter 22 and the tissue 60 adjacent the cavity 62, as is described below. Although other viewing apparatus may be used without departing from the scope of the present invention, in one embodiment the viewing apparatus 70 is a fiber optic scope (e.g., a 3.4 mm Flexible Fiber Optic Nasopharyngoscope, commercially available from Kelleher Medical, Inc. of Richmond, Va.).

To deliver targeted radiation brachytherapy to the tissue 60, when the balloon 32 is in the deflated state a portion of the applicator 20 is inserted into the patient's cavity 62 through its entrance 74, such that the balloon and a portion of the catheter 22 are inserted into the cavity. The radiation source 66 is inserted into the catheter 22 so the radiation source is generally adjacent the balloon. For example, in the exemplary embodiment illustrated in FIG. 3, the wire 68 is inserted into the catheter 22 so the radioactive seed 66 is generally adjacent the balloon 32. In one embodiment, the radiation source 66 is inserted into the catheter 22 prior to insertion of the applicator 20 into the cavity 62. In another embodiment, the radiation source 66 is inserted into the catheter 22 after insertion of the applicator 20 into the cavity 62. In yet another embodiment, the radiation source 66 is inserted into the catheter 22 generally simultaneously with insertion of the applicator 20 into the cavity 62. Once the balloon 32 is received within the cavity 62 and the radiation source 66 is positioned in the catheter 22 adjacent the balloon, pressurized fluid is introduced to the conduit 30 and into the interior 34 of the balloon to inflate the balloon and move it from the deflated state to the inflated state. The balloon 32 is inflated (moved) to an inflated state wherein the radiation source 66 is at a predetermined dwell position, and more specifically a predetermined distance from areas 76 of the tissue 60 targeted for brachytherapy and from areas 78 of the tissue not targeted for brachytherapy. By controlling the predetermined dwell position, a dose distribution of radiation delivered into the tissue 60 can be controlled. More specifically, the amount of radiation delivered to the targeted tissue 76 can be more accurately controlled while facilitating a generally quick fall off dose in the non-targeted tissue 78.

Depending on the type and size of the cavity 62 and/or the desired predetermined dwell position(s) of the radiation source(s), the balloon 32 in the inflated state may completely fill the cavity 62 so the exterior surface 42 of the balloon contacts some or all of the tissue 60, or may only partially fill the cavity as illustrated in FIG. 3. Additionally, depending on the type and size of the cavity 62 and/or the desired predetermined dwell position(s), some or all of the tissue 60 may deform to the shape of the balloon 32 in its inflated state, or portions or all of the balloon in its inflated state may deform to the shape of the cavity. The number of catheters 22 included with the applicator 20 may also depend on the type and size of the cavity 62, the desired predetermined dwell position(s), and/or the size of the entrance 74 to the cavity. For example, when a large area of the tissue 60 is targeted for brachytherapy it may be desirable to include the maximum number of catheters 22 the entrance 74 to the cavity allows to obtain as many different dwell positions for the radiation source(s) 66 as possible.

Additionally, it may be desirable to rotate the balloon 32 to increase the number of dwell positions for the radiation source(s) 66 and thereby further control the dose distribution of radiation delivered into the tissue 60. For example, for large cavities with small entrances, the applicator 20 may include only one catheter 22 so the applicator 20 is more easily and comfortably inserted into the cavity 62, yet the balloon 32 can be rotated to obtain multiple dwell positions for the radiation source(s) in the catheter 22. The viewing apparatus 70 may be used to monitor rotation of the balloon 32 to ensure the radiation source(s) 66 is accurately located at the desired predetermined dwell position(s). Additionally, the viewing apparatus 70 may be used to generally view/monitor/document the tissue 60, including the targeted and non-targeted areas 76, 78, the applicator 20 and its various components, and the brachytherapy procedure being performed on the patient. The viewing apparatus 70 may be positioned anywhere on/in the applicator 20 facilitating its purpose(s). For example, the viewing apparatus 70 may be inserted into a catheter 22 before or after insertion of the applicator 20 and such that the apparatus is positioned in the catheter generally adjacent the balloon 32 for viewing the catheter and the tissue 60.

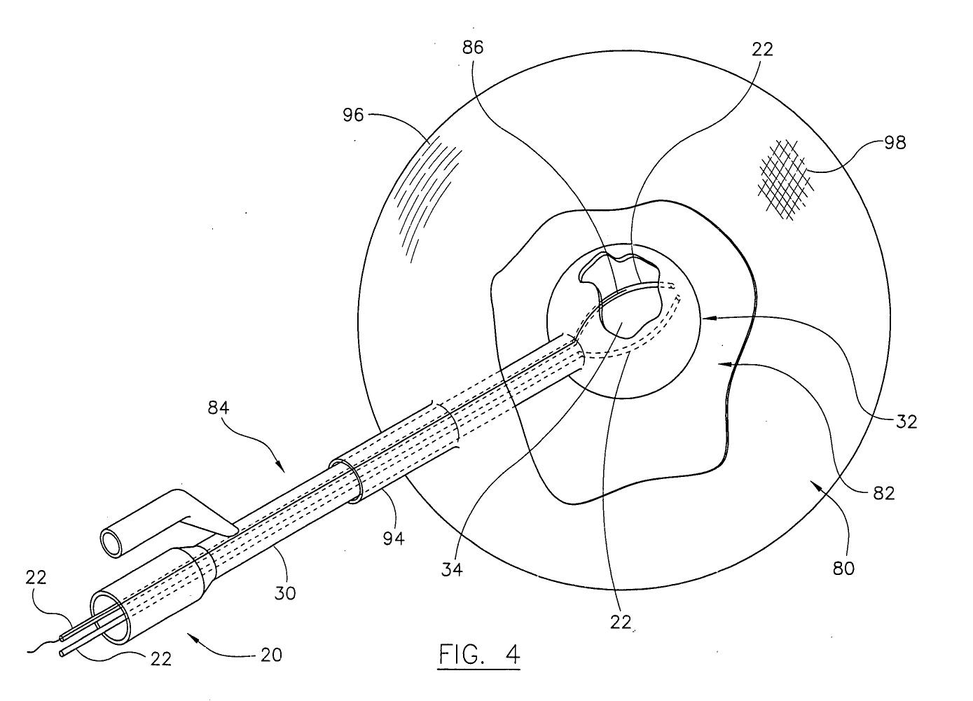

As illustrated in FIG. 4, the applicator 20 described above may be used to deliver targeted thermal therapy to tissue (generally designated by 80) adjacent a patient's cavity (generally designated by 82). More specifically, an applicator system 84 includes the applicator 20 and a heat source (generally designated by 86) in the catheter 22. Each catheter 22 may include any number of heat sources 86. Although other heat sources may be used without departing from the scope of the present invention (e.g., radiofrequency antennas, ultrasound applicators), in the exemplary embodiment illustrated in FIG. 4 the heat source 86 is an antenna 86 configured to emit microwaves into the tissue 80 to heat the tissue. Although other types of antennas may be used without departing from the scope of the present invention (e.g., line dipole or multisection antennas), in one embodiment the antenna 86 is a helical antenna. The antenna 86 is positioned in the catheter 22 such that the antenna emits microwaves generally adjacent the balloon 32. Similar to the applicator system described above and illustrated in FIG. 3, the applicator system 84 may also include a viewing apparatus (not shown) positioned generally adjacent the balloon 32 for viewing the catheter 22 and the tissue 80 adjacent the cavity 82.

To deliver targeted thermal therapy to the tissue 80, when the balloon 32 is in the deflated state a portion of the applicator 20 is inserted into the patient's cavity 82 through its entrance 94, such that the balloon and a portion of the catheter 22 are inserted into the cavity. The heat source 86 is inserted into the catheter 22 so the heat source is generally adjacent the balloon. For example, in the exemplary embodiment illustrated in FIG. 4, the antenna 86 is inserted into the catheter 22 so the antenna emits microwaves generally adjacent the balloon 32. In one embodiment, the heat source 86 is inserted into the catheter 22 prior to insertion of the applicator 20 into the cavity 82. In another embodiment, the heat source 86 is inserted into the catheter 22 after insertion of the applicator 20 into the cavity 82. In yet another embodiment, the heat source 86 is inserted into the catheter 22 generally simultaneous with insertion of the applicator 20 into the cavity 82. Once the balloon 32 is received within the cavity 82 and the heat source 86 is positioned in the catheter 22 adjacent the balloon, pressurized fluid is introduced to the conduit 30 and into the interior 34 of the balloon to inflate the balloon and move it from the deflated state to the inflated state. The balloon 32 is inflated to an inflated state wherein the heat source 86 is at a predetermined dwell position, and more specifically a predetermined distance from areas 96 of the tissue 80 targeted for thermal therapy and from areas 98 of the tissue not targeted for thermal therapy. By controlling the predetermined dwell position, a temperature increase of the targeted tissue 96 and the non-targeted tissue 98 can be controlled.

Depending on the type and size of the cavity 82 and/or the desired predetermined dwell position(s) of the heat source(s), the balloon 32 in the inflated state may completely fill the cavity 82 such that the exterior surface 42 of the balloon contacts some or all of the tissue 80, or may only partially fill the cavity as illustrated in FIG. 4. Additionally, depending on the type and size of the cavity 82 and/or the desired predetermined dwell position(s), some or all of the tissue 80 may deform to the shape of the balloon 32 in its inflated state, or portions or all of the balloon in its inflated state may deform to the shape of the cavity. The number of catheters 22 included with the applicator 20 may also depend on the type and size of the cavity 82, the desired predetermined dwell position(s), and/or the size of the entrance 94 to the cavity. For example, when a large area of the tissue 80 is targeted for thermal therapy it may be desirable to include the maximum number of catheters 22 the entrance 94 to the cavity allows to obtain as many different dwell positions for the heat source(s) 86 as possible.

Similar to the applicator system described above and illustrated in FIG. 3, it may be desirable to rotate the balloon 32 to increase the number of dwell positions for the heat source(s) 86. As described above with regard to FIG. 3, a viewing apparatus may be used to monitor rotation of the balloon 32 as well as to generally view/monitor the tissue 80, including the targeted and non-targeted areas 96, 98, as well as applicator 20 and its various components.

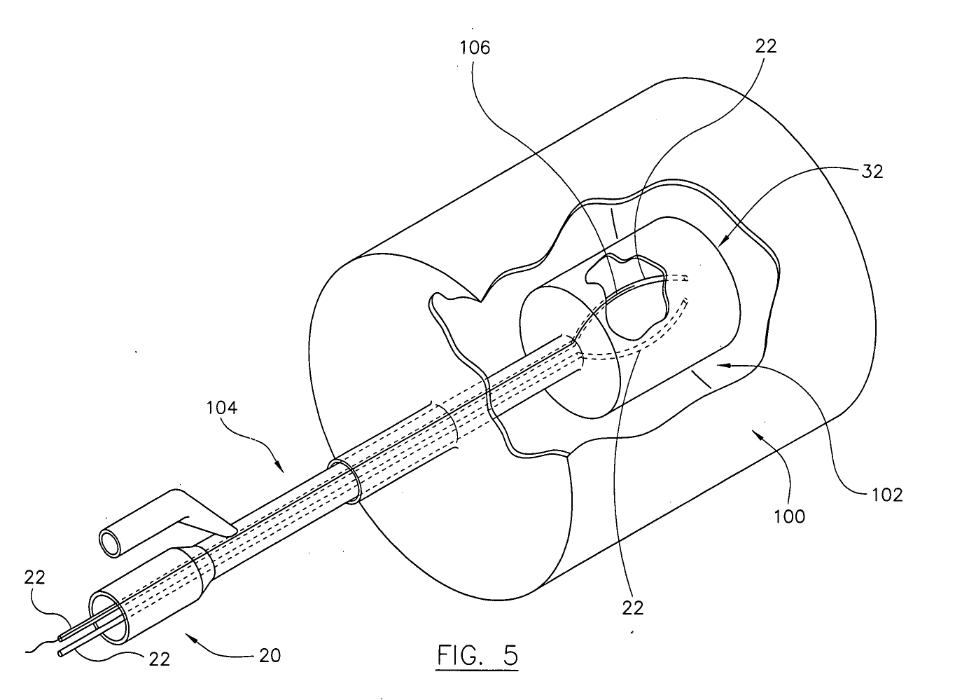

As illustrated in FIG. 5, the applicator 20 described above may be used to facilitate the delivery of external beam radiation and/or external thermal therapy to tissue (generally designated by 100) adjacent the patient's cavity (generally designated by 102). More specifically, an applicator system 104 includes the applicator 20 and a radio opaque marker (generally designated by 106) in the catheter 22. Each catheter 22 may include any number of radio opaque markers 106. Although other radio opaque markers 106 may be used without departing from the scope of the present invention (e.g., cerrobend, steel), in the exemplary embodiment illustrated in FIG. 5 the radio opaque marker 106 is formed from lead. The marker 106 is positioned in the catheter 22 so that the marker is generally adjacent the balloon 32. When an x-ray is taken of the patient's cavity 102, the marker 106 can then be used to mark the location of the balloon 32 to facilitate the delivery of external beam radiation and/or external thermal therapy to a predetermined area of the tissue 100.

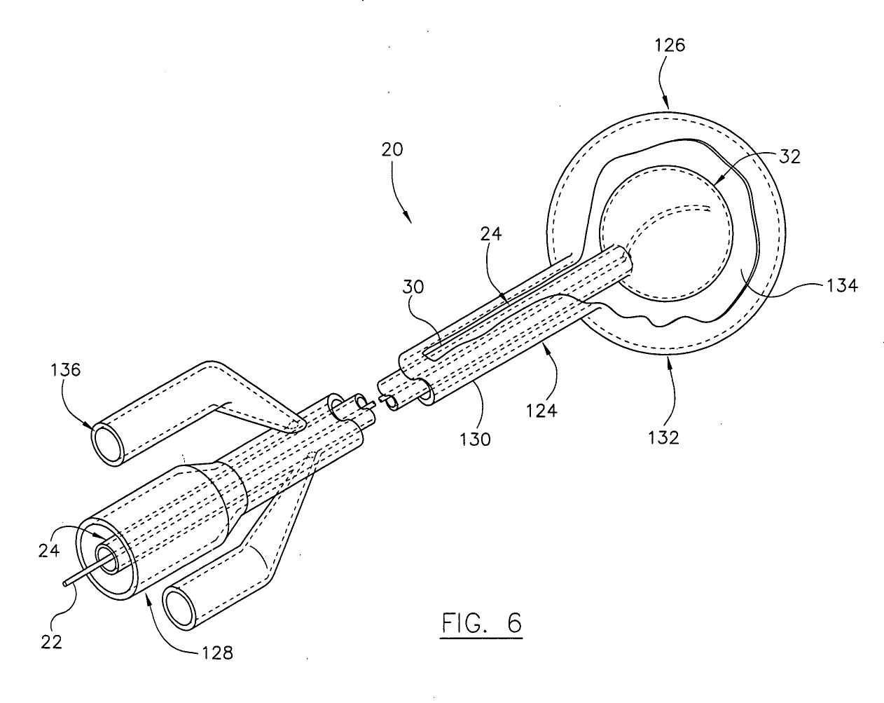

As illustrated in FIG. 6, in an alternative embodiment the applicator 20 includes a body (generally designated by 124) in addition to the body 24. The body 124 has a first end (generally designated by 126), a second end (generally designated by 128), a conduit 130 extending between the first end and the second end, and a balloon (generally designated by 132). Either of the conduit 30 and the conduit 130 may be referred to herein as a first conduit or a second conduit. Additionally, either of the balloon 32 and the balloon 132 may be referred to herein as a first balloon or a second balloon. As with the body 24, the balloon 132 is adapted for introduction to a patient's cavity. More specifically, the balloon 132 has a deflated state (not shown) in which the balloon 132 and the first end 126 of the body 124 are adapted for insertion into the cavity through its entrance. Additionally, at least a portion of the conduit 130 may also be adapted for insertion through the entrance and into the cavity. The first end 126 of the body 124, the balloon 132 in its deflated state, and where applicable all or a portion of the conduit 130, are sized and shaped appropriately for insertion into the particular cavity (e.g., bladder) through its entrance (e.g., urethra). The body 124 is positioned relative to the body 24 so the balloon 132 is adjacent the balloon 32, such that the balloon 132 is adapted for introduction to the patient's cavity generally simultaneous with the balloon 32. In one embodiment, as illustrated in FIG. 6, the body 124 surrounds the body 24 such that the balloon 132 surrounds the balloon 32.

As illustrated in FIG. 6, the balloon 132 also has an inflated state in which the balloon is enlarged for at least partially filling the cavity. The conduit 130 is in fluid communication with an interior 134 of the balloon 132 for introducing pressurized fluid to the interior of the balloon to move the balloon 132 from the deflated state to the inflated state. Pressurized fluid is introduced into the conduit 130 through an opening (generally designated by 136) within the body 124 in fluid communication with the conduit. Although any suitable fluid (e.g., saline) may be introduced into the conduit 130 and the interior 134 of the balloon to move the balloon from the deflated state to the inflated state without departing from the scope of the present invention, in one embodiment air is used to move the balloon from the deflated state to the inflated state. As illustrated in FIG. 6, the balloon 132 defines the first end 126 of the body 124. However, the balloon 132 may be suitably positioned anywhere along the body 124 such that the balloon 132 is adjacent the balloon 32 and is adapted for insertion into the cavity and movement to the inflated state once received within the cavity. Additionally, as with the balloon 32, in the inflated state the balloon 132 may be suitably shaped for the particular cavity. The particular size (e.g., radius) of the balloon 132 in the inflated state may also vary for the particular cavity. Additionally, the balloon 132 may be inflatable to a variety of sizes and/or shapes such that the inflated state of the balloon may comprise a plurality of states each having a different size and/or shape.

Similar to the body 24, the body 124 may be formed from any suitable material(s), for example rubber and/or plastic. Although different portions of the body 124 may be formed from different materials, in one embodiment the entirety of the body is formed from one material. The portions of the body 124 adapted for introduction to the patient's cavity may be formed from any material suitable for use within the cavity, so that such portions do not damage tissue adjacent the cavity and/or injure/infect the patient. Additionally, in one embodiment at least a portion of the body 124 (e.g., at least a portion of the conduit 130 and/or the balloon 132) is formed from a transparent material to facilitate use of a viewing apparatus (not shown in FIG. 6) with the applicator 20, as is described in more detail above.

This alternative embodiment of the applicator 20 facilitates even more control over an accurate predetermined dwell position of the radiation source(s) 66 (FIG. 3) and/or the heat source(s) 86 by using two separate balloons 32, 132 to control the position of catheter 22, and therefore the radiation source(s) and/or the heat source(s), and the position of the tissue adjacent the cavity, respectively.

Although each of the applicator systems described and illustrated herein are described and illustrated separately, it should be understood that the systems may be used in combination to perform a combination of targeted radiation brachytherapy and/or targeted thermal therapy, and/or to facilitate external beam radiation. For example, an applicator of the present invention may include a catheter having a radiation source therein, a catheter having a heat source therein, and/or a catheter having a radio opaque marker therein. Additionally, an applicator of the present invention may include a catheter having one or more of a radiation source, a heat source, and a radio opaque marker therein. Accordingly, a single applicator of the present invention may be used to simultaneously perform a combination of targeted radiation brachytherapy and/or targeted thermal therapy, and/or to facilitate external beam radiation and/or external thermal therapy.

As used herein, the term “cavity” includes any cavity of any animal where it is desired to deliver targeted radiation brachytherapy to tissue adjacent the cavity.

The above-described applicator and applicator systems are cost-effective and reliable for performing targeted radiation brachytherapy and targeted thermal therapy, and for facilitating external beam radiation. More specifically, the applicator and applicator systems of the present invention may facilitate access to previously inaccessible organs and cavities for targeted radiation brachytherapy and targeted thermal therapy such as, for example, the bladder, the rectum, the esophagus, the stomach, the bronchus, nasopharynx, and the nasal cavity. Additionally, the present invention can be rotated to allow an almost unlimited number of potential dwell positions for radiation and/or heat sources, and a viewing apparatus may be used along with the applicator to ensure accurate positioning of the radiation and/or heat sources, as well as generally monitoring the procedure being performed. Furthermore, access to most cavities is no more invasive than placement of a Foley catheter, which may allow for outpatient treatment with minimum or no analgesia.

Exemplary embodiments of applicator systems are described above in detail. The systems are not limited to the specific embodiments described herein, but rather, components of each system may be utilized independently and separately from other components described herein. Each applicator system component can also be used in combination with other applicator system components.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the invention, it is intended that all matter contained in the above description or shown in the accompanying drawings shall be interpreted as illustrative and not in a limiting sense.

1. An applicator for delivering targeted radiation brachytherapy to tissue adjacent a cavity of a patient, said applicator comprising:
a balloon adapted for introduction to the cavity of the patient, said balloon having a deflated state in which the balloon is adapted for insertion into the cavity and an inflated state in which the balloon is enlarged for at least partially filling the cavity of the patient, said balloon moving from said deflated state to the inflated state upon introduction of pressurized fluid to an interior of the balloon;
a conduit in fluid communication with the interior of the balloon for introducing pressurized fluid to the interior of the balloon to move the balloon from the deflated state to the inflated state; and
a catheter extending over at least a portion of the balloon for delivering radiation from a radiation source to the tissue adjacent the cavity.

2. An applicator in accordance with claim 1 wherein the catheter extends through the conduit and along an interior surface of the balloon.

3. An applicator in accordance with claim 1 wherein the balloon is generally cylindrical when the balloon is in the inflated state.

4. An applicator in accordance with claim 1 wherein the balloon is generally spherical when the balloon is in the inflated state.

5. An applicator in accordance with claim 1 wherein the conduit is at least partially transparent.

6. An applicator in accordance with claim 1 wherein the conduit is a first conduit, the balloon is a first balloon, and the applicator further comprises:
a second balloon adjacent the first balloon, said second balloon being adapted for introduction to the cavity of the patient simultaneous with the first balloon, said second balloon having a deflated state in which the second balloon is adapted for insertion into the cavity and an inflated state in which the second balloon is enlarged for at least partially filling the cavity of the patient, said second balloon moving from the deflated state to the inflated state upon introduction of pressurized fluid to an interior of the second balloon; and
a second conduit in fluid communication with the interior of the second balloon for introducing pressurized fluid to the interior of the second balloon to move the second balloon from the deflated state to the inflated state.

7. An applicator in accordance with claim 6 wherein the second balloon surrounds the first balloon.

8. An applicator in accordance with claim 1 wherein the radiation source comprises a radioactive seed attached to a wire positioned in the catheter such that the seed is generally adjacent the balloon.

9. An applicator in accordance with claim 8 wherein the balloon is at least partially transparent, and said applicator further comprises a viewing apparatus generally adjacent the balloon for viewing the catheter and the tissue adjacent the cavity.

10. An applicator system for delivering targeted thermal therapy to tissue adjacent a cavity of a patient, said applicator system comprising:
a balloon adapted for introduction to the cavity of the patient, said balloon having a deflated state in which the balloon is adapted for insertion into the cavity and an inflated state in which the balloon is enlarged for at least partially filling the cavity of the patient, said balloon moving from the deflated state to the inflated state upon introduction of pressurized fluid to an interior of the balloon;
a conduit in fluid communication with the interior of the balloon for introducing pressurized fluid to the interior of the balloon to move the balloon from the deflated state to the inflated state; and
a catheter extending over at least a portion of the balloon, said conduit having a heat source therein for delivering heat to the tissue adjacent the cavity.

11. An applicator system in accordance with claim 10 wherein the heat source comprises an antenna configured to emit microwaves into the tissue adjacent the cavity thereby to heat the tissue.

12. An applicator system in accordance with claim 11 wherein the antenna is a helical antenna.

13. An applicator system in accordance with claim 10 wherein the balloon is generally transparent, and said applicator system further comprises a viewing apparatus generally adjacent the balloon for viewing the catheter and the tissue adjacent the cavity.

14. An applicator system in accordance with claim 10 wherein the conduit is a first conduit, the balloon is a first balloon, and said applicator system further comprises:
a second balloon adjacent the first balloon, said second balloon being adapted for introduction to the cavity of the patient simultaneous with the first balloon, said second balloon having a deflated state in which the second balloon is adapted for insertion into the cavity and an inflated state in which the second balloon is enlarged for at least partially filling the cavity of the patient, said second balloon moving from the deflated state to the inflated state upon introduction of pressurized fluid to an interior of the second balloon; and
a second conduit in fluid communication with the interior of the second balloon for introducing pressurized fluid to the interior of the second balloon to move the second balloon from the deflated state to the inflated state.

15. A method of delivering targeted radiation brachytherapy to tissue adjacent a cavity of a patient using an applicator comprising a balloon having a deflated state in which the balloon is adapted for insertion into the cavity of the patient and an inflated state in which the balloon is enlarged for at least partially filling the cavity, said method comprising:
attaching a catheter to the balloon for movement with the balloon;
inserting the balloon and the catheter into the cavity when the balloon is in the deflated state;
inserting a radiation source into the catheter so the radiation source is generally adjacent the balloon;
inflating the balloon within the cavity so the radiation source is a predetermined distance from the tissue adjacent the cavity; and
controlling a dose distribution of radiation delivered into the tissue adjacent the cavity by the radiation source by controlling the predetermined distance of the radiation source from the tissue.

16. A method in accordance with claim 15 further comprising rotating the balloon within the cavity to further control the dose distribution of radiation delivered into the tissue by the radiation source.

17. A method in accordance with claim 15 wherein the balloon is a first balloon, the applicator further comprises a second balloon adjacent the first balloon, the second balloon has a deflated state in which the second balloon is adapted for insertion into the cavity of the patient and an inflated state in which the second balloon is enlarged for at least partially filling the cavity, said step of inserting the balloon and the catheter into the cavity when the balloon is in the deflated state comprises inserting the second balloon into the cavity simultaneously with the first balloon when the second balloon is in the deflated state, and said step of inflating the balloon within the cavity comprises inflating the second balloon so the second balloon at least partially fills the cavity.

18. A method of delivering targeted thermal therapy to tissue adjacent a cavity of a patient using an applicator comprising a balloon having a deflated state in which the balloon is adapted for insertion into the cavity of the patient and an inflated state in which the balloon is enlarged for at least partially filling the cavity, said method comprising:
attaching a catheter to the balloon for movement with the balloon;
inserting the balloon and the catheter into the cavity when the balloon is in the deflated state;
inserting a heat source into the catheter so the heat source is generally adjacent the balloon;
inflating the balloon within the cavity so the heat source is a predetermined distance from the tissue adjacent the cavity; and
controlling a temperature increase of the tissue adjacent the cavity by controlling the predetermined distance of the heat source from the tissue.

19. A method in accordance with claim 18 further comprising rotating the balloon within the cavity to further control the temperature increase of the tissue.

20. A method in accordance with claim 19 wherein the balloon is a first balloon, the applicator further comprises a second balloon adjacent the first balloon, the second balloon has a deflated state in which the second balloon is adapted for insertion into the cavity of the patient and an inflated state in which the second balloon is enlarged for at least partially filling the cavity, said step of inserting the balloon and the catheter into the cavity when the balloon is in the deflated state comprises inserting the second balloon into the cavity simultaneously with the first balloon when the second balloon is in the deflated state, and said step of inflating the balloon within the cavity comprises inflating the second balloon so the second balloon at least partially fills the cavity.

21. An applicator system for facilitating the delivery of at least one of external beam radiation and external thermal therapy to tissue adjacent a cavity of a patient, said applicator system comprising:
a balloon adapted for introduction to the cavity of the patient, said balloon having a deflated state in which the balloon is adapted for insertion into the cavity and an inflated state in which the balloon is enlarged for at least partially filling the cavity of the patient, said balloon moving from the deflated state to the inflated state upon introduction of pressurized fluid to an interior of the balloon;
a conduit in fluid communication with the interior of the balloon for introducing pressurized fluid to the interior of the balloon to move the balloon from the deflated state to the inflated state; and
a catheter extending over at least a portion of the balloon, said catheter having a radio opaque marker therein adjacent the balloon for marking a position of the balloon when the balloon is received within the cavity.
